(19) United States
(12) Patent Application Publication
(10) Pub. No.: US 2015/0190345 A1
(43) Pub. Date: Jul. 9, 2015

(54) Controlled Release Formulations of Lorazepam

(71) Applicants: Douglas A. Saltel, Austin, TX (US); Michael Vachon, Quebec (CA)

(72) Inventors: Douglas A. Saltel, Austin, TX (US); Michael Vachon, Quebec (CA)

(21) Appl. No.: 14/150,493

(22) Filed: Jan. 8, 2014

(51) Int. Cl. A61K 9/20; A61K 31/5513

(52) U.S. Cl. CPC A61K 9/2031; A61K 31/5513; A61K 9/2018; A61K 9/2009

(57) ABSTRACT

Controlled release of lorazepam can provide enhanced dosing options including once daily dosing that provides 24 hour therapeutic effect under steady state conditions. The pharmaceutical composition can provide substantially zero order release and 90% release within 7 to 12 hours in a pharmaceutical dissolution test. The release can be achieved using polyethylene oxide as a matrix polymer.

The present application claims the benefit of priority under 35 U.S.C. §119(e) from prior U.S. provisional patent application No. 61/750,792, filed on Jan. 9, 2013 and from prior U.S. provisional patent application No. 61/762,836, filed on Feb. 8, 2013; the entire contents of each provisional application being incorporated herein by reference.

### BACKGROUND OF THE INVENTION

The present invention relates to controlled release formulations of lorazepam and to methods of treating patients with a once-a-day dose of lorazepam.

Lorazepam is the generic name for the active pharmaceutical ingredient 7-chloro-5-(2-chlorophenyl)-1,3-dihydro-3-hydroxy-2H-1,4-benzodiazepin-2-one, which has the following structure:

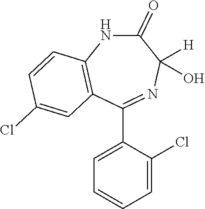 [SMILES: O=C1Nc2ccc(Cl)cc2C(c2ccccc2Cl)=NC1O]

Like other benzodiazepines, lorazepam has CNS activity and has proven to be a useful treatment for anxiety related disorders, such as: General Anxiety Disorder or Anxiety associated with Major Depression and others. It is almost insoluble in water. This compound was disclosed in U.S. Pat. No. 3,296,249.

Lorazepam has been sold commercially under the brand name ATIVAN® (originally by Wyeth, now by Valeant Intl) in the form of an oral immediate release tablet. The tablets contain 0.5 mg, 1 mg, or 2 mg of lorazepam and are usually administered two or three times a day (b.i.d and t.i.d, respectively) to achieve a total dose of 2 to 6 mg/day, though doses from 1 to 10 mg/day can also be used. According to the U.S. package insert material for ATIVAN®: “For anxiety, most patients require an initial dose of 2 to 3 mg/day given b.i.d. or t.i.d.” The peak plasma concentrations (Cmax) typically occur about 2 hours (Tmax) after oral administration. Lorazepam has, according to the package insert, a half-life in human plasma of about 12 hours.

While the immediate release tablets of lorazepam, with a multi-dose per day regimen, have been available for several decades, thus far no once-a-day dosage form has been commercially introduced. Such a dosage form is often desirable. Besides the benefit of convenience, a sustained release version that could provide 24 hour therapeutic effect, but with lower peak plasma concentration levels than the immediate release tablet, may reduce side effects. For this reason, Abrams et al. investigated a sustained release tablet containing 2 mg of lorazepam and compared it to a 2 mg dose of lorazepam immediate release tablets (2×1 mg tablets). S. M. L. Abrams et al., “Pharmacodynamic and Pharmacokinetic Comparison of Two Formulations of Lorazepam and Placebo,” Human Psychopharmacology, Vol. 3, 133-138 (1988). The sustained release tablet had, as expected, a longer Tmax (median 8 hours) and a lower Cmax (12 ng/ml) than the immediate release tablets (2 hours and 22 ng/ml, respectively). But the relative bioavailability was reduced in the sustained release tablet such that after 30 hours the AUC was only about 85% of the AUC achieved with the immediate release tablets. Abrams et al. also noted that “the [serum] concentrations of both formulations were similar between 10 and 30 h[ours].” Thus, despite providing some delay in the rise of lorazepam serum concentrations and a lower Cmax, the sustained release tablet apparently did not serve to extend the therapeutic duration of lorazepam beyond that achieved with immediate release tablets.

Regarding the tablet formulation, Abrams et al. does not disclose the design or excipients used in making the sustained release tablet. From the plasma concentration curve, the sustained release tablet appears to have been a first order release tablet, though the kinetics are not reported.

The long half-life of lorazepam in blood plasma makes it a classically disregarded candidate for the development of a once daily formulation. Also, a drug product that provides 24 hour therapy from two doses per day, as opposed to three or more doses per day, is generally considered to have achieved the majority of patient compliance benefits. If a single daily dose formulation was desired, an immediate release lorazepam tablet could be used to provide a complete daily dose because of the long half-life of lorazepam in blood plasma. But administering a complete daily dose in a single immediate release dosage form would increase the Cmax and the peak-trough variations (concentration differences between Cmax and Cmin) beyond those attained in conventional b.i.d. administration (i.e., twice-daily dosing), and thus would likely increase the risk of drug related adverse events, i.e., side effects. Using a sustained release formulation can reduce the rate of increase in plasma drug concentration and the value of Cmax, but runs the risk of sub-therapeutic plasma concentration levels, especially near the end of the dosing cycle, and/or lower overall drug exposure than the current b.i.d. immediate release tablet regimen.

A lorazepam formulation that provides a sustained release profile with the potential for an effective and well tolerated once daily dosing regimen would be advantageous.

### SUMMARY OF THE INVENTION

The present invention relates to controlled release lorazepam compositions. A first aspect of the invention relates to a pharmaceutical composition, comprising 0.5 to 10 mg of lorazepam in combination with sufficient pharmaceutically acceptable excipients to provide a solid oral dosage form having controlled release of said lorazepam; wherein said controlled release of lorazepam is:

(1) substantially zero order release; and
(2) the release of lorazepam reaches 90% within the time range of 7 to 12 hours. The controlled release parameters are determined in a pharmaceutical dissolution test comprising a buffer of pH 6.8. Preferably the routine of once-daily administration of the composition can provide the patient with 24 hours of therapeutic effect.

Another aspect of the invention relates to a controlled release tablet, which comprises 0.5 to 10 mg of lorazepam dispersed in a controlled release matrix, wherein said matrix comprises polyethylene oxide. Often the polyethylene oxide has a molecular weight of 900,000 to 2,000,000 such as about 1,000,000. Preferably the tablet exhibits sufficient controlled and complete release of lorazepam to facilitate a once daily dosing regimen that provides 24 hours of therapeutic effect.

A further aspect of the invention relates to a method of treating a lorazepam-treatable condition in a patient, which comprises administering once a day to a patient in need thereof a controlled release lorazepam composition that contains 0.5-10 mg of lorazepam that provides 24 hour therapeutic effect during steady state conditions. The composition exhibits the zero order controlled release as described above and/or is a polyethylene oxide matrix tablet as described above. Typical lorazepam-treatable conditions include anxiety disorders such as Generalized Anxiety Disorder and anxiety associated with major depression, but is not limited thereto.

### BRIEF DESCRIPTION OF THE DRAWING

FIG. 1 represents the dissolution of the tablets made in the Example using a pharmaceutical dissolution test.

### DETAILED DESCRIPTION OF THE INVENTION

The present invention relates to lorazepam formulations. Providing controlled release lorazepam suitable for once daily dosing is problematic. Slowing the rate of drug release presents the risk of a sub-therapeutic plasma concentration, especially in the hours before or after the daily dose is administered. This period of low plasma concentration can leave a patient susceptible to break-through anxiety; e.g., the anxiety breaks through the effect of the drug. Giving the drug faster and/or giving more drug to compensate for the low plasma levels and avoiding break-through anxiety increases the risk of adverse events (commonly referred to as side effects) such as sedation, dizziness, memory impairment, etc. Indeed, but for the nearly certain increase in adverse effects as a result of the rapid and high peak plasma concentration, a large enough immediate release dose of lorazepam could provide therapeutic levels for 24 hours by simply exploiting the inherent long plasma half-life of the drug. Thus, patient safety issues preclude the use of large single immediate release doses as not being practical for lorazepam.

Commonly, an oral controlled release formulation exhibits first order release. This means that the rate of drug release is proportional to the amount of drug in the formulation. As more drug is released, the rate of release decreases. Applying mathematical models, it was discovered that a first order release profile was not well suited for developing a controlled release lorazepam tablet. Specifically the risk of either sub-therapeutic plasma levels late in the dosing interval or adverse events was too great when attempting to design a first order release system. Instead, it was discovered that a zero order release of lorazepam over an extended period of time could provide safe and effective plasma concentrations of lorazepam.

Accordingly, one aspect of the present invention relates to a pharmaceutical composition comprising 0.5 to 10 mg of lorazepam in combination with sufficient pharmaceutically acceptable excipients to provide a solid oral dosage form having controlled release of lorazepam. The controlled release exhibits the following parameters: (1) substantially zero order release and (2) the release of lorazepam reaches 90% within the time range of 7 to 12 hours. These controlled release parameters are determined in a pharmaceutical dissolution test comprising buffer of pH 6.8. For clarity, a “pharmaceutical dissolution test” is any in vitro dissolution test carried out under pharmaceutically reasonable conditions. As is well known in the art, different formulations are often tested under different conditions, such as basket apparatus or a paddle apparatus, varying paddle speeds (e.g., 50 rpm, 60 rpm, or 75 rpm), etc., in order to obtain meaningful data. Typically dissolution testing is used to model in vivo release and/or to differentiate improperly made formulations from correctly manufactured ones. Developing a suitable set of conditions for dissolution is a matter of routine practice and skill. For the present invention, the pharmaceutical dissolution test must include the use of a pH 6.8 buffer. The entire test need not be performed at pH 6.8 or in buffer, but at least a portion of the test must comprise the use of pH 6.8 buffer; i.e., two phase testing, variable pH testing, etc., are permissible. Other than the requirement that an aqueous buffer solution of pH 6.8 be used at some point, the other conditions can be varied as appropriate. Tests that use unreasonable conditions, i.e., those that would give no meaningful data and/or are designed specifically to not meet the release parameters of the present invention are excluded. Thus, any dissolution test, which includes a pH 6.8 buffer, that a regulatory authority, such as the U.S. FDA, finds acceptable to support a filing for a controlled release lorazepam formulation, is a dissolution test conducted under pharmaceutically reasonable conditions and is therefore a “pharmaceutical dissolution test” for purposes of the present invention. In that several pharmaceutically reasonable dissolution tests may exist, the release parameters of the present invention need only be satisfied in one such test—not in every possible test and regardless if not satisfied in another test. The pH 6.8 buffer is typically a phosphate buffered aqueous solution and may contain additional ingredients such as in forming a complete simulated intestinal fluid (SIF) with (or without) enzymes as is known in the art. A typical pharmaceutical dissolution test uses 500 ml or 900 ml of media at 37° C. with paddles at an rpm of 50 to 100 such as 60 or 75, but is not limited thereto as explained above. When the pharmaceutical composition releases lorazepam substantially independently of pH, then a single media is often suitable, e.g., the whole test can be conducted with pH 6.8 buffer. If the composition has a pH-sensitive release (e.g., the rate of release is affected by the pH of the media), then different media are often desired in making a suitable, pharmaceutically acceptable test. For example, a two phase test may be used wherein the initial phase is carried out in a lower pH environment followed by the higher pH 6.8 buffer environment. The first phase is often 0.1 N HCl and/or simulated gastric fluid (SGF) and last typically between 1-3 hours, often 1 or 2 hours. The media is changed to start the second phase in pH 6.8 buffer and the test carried to completion. The initial phase could be a higher pH such as around pH 4 to simulate a fed stomach, etc. as is well known in the art. In like manner to the two phase test having low and moderate pH, multi-phase tests could be adopted having 3, 4, or more different media phases. More recently, variable pH dissolution testing has become more technologically available wherein the number of stages can be very great so as to approximate constant pH change over all or a portion of the dissolution test.

The percentage of lorazepam released during the pharmaceutical dissolution test is stated with respect to the nominal or label amount, as is conventional in the art. Thus, the release of 1 mg lorazepam from a 2 mg tablet is reported as 50% release, even if the tablet is discovered to only contain 1.94 mg of lorazepam instead of the intended 2.0 mg. In a well-controlled process, the actual assayed amount is generally within +/−5% of the label amount. The percentage of release at a point in time refers to the cumulative release up to that point in time, as per the conventional usage of these terms in the art. The amount of lorazepam released from the beads (i.e., dissolved into the dissolution media), can be determined by ordinary methods using routine skill.

The pharmaceutical composition of the invention provides controlled release of lorazepam. As used herein, “controlled release” means any type of prolonged release of the drug beyond immediate release and is not intended to denote any other quality or characteristic of the release. Unless otherwise indicated, controlled release is used synonymously with extended release and sustained release.

The controlled release for this aspect of the present invention has two characterizing parameters: substantially zero order release and a specified time to completion of release. Zero order release, as is well known in the art, means that the rate of release is constant over time and not proportional to the amount of drug in the formulation. The dissolution curve of a perfect zero order release profile, plotted as cumulative drug release (Y axis) vs. time (X axis), would be a straight line of positive slope. This is in contrast to the more common first order release profile, where the initial rate of release is high but slows down over time as the concentration of drug in the dosage form is diminished.

The present invention does not require a perfect zero order release, merely a substantially zero order release. Workers skilled in the art are accustomed to categorizing release as zero order or first order, etc. A release curve is substantially zero order when that is the best category or description; i.e., declaring it first order would be less accurate or less correct. Sometimes a worker skilled in the art will classify the type of release for a portion of the dissolution release curve. For example, one portion exhibits zero order release and another exhibits first order. In this situation, the zero order part should comprise at least 50%, more typically at least 60%, and often at least 70% of the amount of lorazepam that is released, in order for the overall release to be considered substantially zero order. For clarity, the percentage is not necessarily from zero, but is a continuous portion of the curve, e.g., zero order release from 10% to 60% release of lorazepam means that 50% of the lorazepam was released under zero order. Of course, higher portions of zero order or substantially zero order release are generally preferred, including at least 75%, at least 80%, at least 85%, and at least 90%.

In more quantitative terms, a dissolution curve shows substantially zero order release when the overall curve is fairly linear or a significant portion is highly linear. In general the correlation coefficient for the zero order release linear equation Qₜ=Kₒt+Qₒ is at least 0.85, more typically at least 0.90, and often at least 0.95 including 0.97, 0.98, and 0.99 (Qₜ is the quantity of released drug, Qₒ is the initial quantity of released drug, Kₒ is the zero order rate constant, and t is time). Alternatively, a release curve may be considered to have zero order and first order release phases. In such an event, a curve is substantially zero order when at least 6 continuous hours, preferably at least 7 continuous hours, and more likely at least 8 continuous hours are clearly zero order release. Such clear phases of zero order release typically have a correlation coefficient of at least 0.95, more preferably at least 0.99.

Another mathematical model for determining substantially zero order release relies on the equation Qₜ/Q_{T}=ktⁿ wherein Qₜ is the quantity of drug released at time t, Q_{T} is the total quantity of drug in the dosage form, k is the constant, and n is the release kinetics exponent. When n is about 1, then the release is zero order release. Substantially zero order release includes n having a value of 0.6 to 1.2, more typically from 0.7 to 1.05, and often from 0.8 to 1.

For purposes of the present invention, a substantially zero order release is one that meets any of the above qualitative or quantitative criteria or definitions of substantially zero order release, unless otherwise noted. Often a substantially zero order release will meet more than one criteria/definition, but such is not required.

The pharmaceutical composition according to this embodiment of the invention not only has a substantially zero order release, but also achieves 90% release of the lorazepam within the range of 7 to 12 hours, more typically within 7.5 to 11 hours. For clarity, the percentage is based on the stated or nominal value of lorazepam in the starting composition, as per the custom in the art. Releasing drug too rapidly can increase the risk of adverse events. Thus, the 90% released point should not occur too soon. But delaying release too long can increase the risk of incomplete absorption and/or sub-therapeutic levels. Accordingly, 90% release generally does not occur until 7 hours or later, more typically not earlier than 7.5 hours, and often not earlier than 8 hours. Conversely, 90% released generally occurs not later than 12 hours, typically before 11 hours and often by 10 hours. Typically at 2 hours in the pharmaceutical dissolution test, not more than 40% of the lorazepam has been released and preferably not more than 35%, and in some embodiments not more than 30% has been released.

The pharmaceutical composition contains 0.5 to 10 mg of lorazepam. Lorazepam and its synthesis are well known and the drug is generally commercially available. Lorazepam can be amorphous or crystalline. Though it has a diazepine ring nitrogen that could be used for forming a salt, typically lorazepam is used as a non-salt or free base. For purposes of the present invention, “lorazepam” is intended to embrace all such pharmaceutical forms of lorazepam including pharmaceutically acceptable salts, crystalline forms thereof including hydrates and solvates, and amorphous forms, unless noted otherwise. The amount of lorazepam in the composition is 0.5 to 10 mg in conformance with the normal total daily dose range. Typical amounts for commercial reasons are often from 1 to 4 mg including 1 mg, 2 mg, 2.5 mg, 3 mg, and 4 mg; though each integer from 1 to 10, inclusive, also represents suitable specific dose amounts.

The pharmaceutical composition contains lorazepam in combination with sufficient pharmaceutically acceptable excipients to provide a solid oral dosage form having controlled release of the lorazepam. Pharmaceutical excipients for making a solid oral dosage form including excipients that alone, or in combination, create controlled release are well known in the art. The structure of solid oral dosage forms that exhibit controlled release are also well known including tablets, beads or pellets in a capsule, osmotic devices, etc.

To obtain substantially zero order release from a controlled release formulation, several design approaches are known. For example, the osmotic device developed by ALZA Corporation is purported to provide constant release rates over time, i.e., zero order release. These osmotic devices, which can have the external appearance of a tablet, generally comprise a chamber having the drug in the interior. The walls of the chamber take up water but do not let the drug out. The sole means of escape for the drug is through a hole (passageway) in the chamber, typically bored with a drill or laser. The interior often contains additional excipients such as to absorb water, to gel, etc. The osmotic pressure developed within the chamber serves to drive the drug out of the passageway in a pH independent and relatively constant rate, which is controlled by the excipients and the size of the passageway. A few examples of such technology include U.S. Pat. No. 3,786,813; U.S. Pat. No. 3,845,770; and U.S. Pat. No. 4,624,847. A variation on this concept where a plug is removed in vivo to form a hole in a chamber has also been proposed as a suitable device for achieving zero order release in U.S. Pat. No. 7,195,778. A compact that uses hydrostatic pressure and without a chamber and hole has been proposed to provide zero order release in U.S. Pat. No. 8,231,897. Another design approach uses multi-layer beads. Various tablets have been proposed for achieving (substantially) zero order release. In general the tablets rely on an erodible matrix such that release is (primarily) dissolution controlled instead of diffusion controlled. Erodible matrix tablets include biodegradable matrix types and gelling polymer matrix-types. The gelling polymer first swells and then undergoes dissolution/erosion at the surface boundary layer. Gelling polymers include hydroxypropyl methylcellulose (HPMC), poly(ethylene oxide) (PEO) which is sometimes also referred to as polyethylene glycol, polyvinyl alcohol (PVA), and various acrylate polymers often sold commercially under the brand name EUDRAGIT. Examples of the use of gelling polymers as a matrix for controlled release tablets include U.S. Pat. No. 4,361,545; U.S. Pat. No. 5,009,895; U.S. Pat. No. 5,945,125; and U.S. Pat. No. 6,703,045; each patent purporting to obtain zero order release.

For ease of manufacture, a tablet is the preferred solid oral dosage form. Typically a tablet of the invention comprises lorazepam dispersed in a gelling polymer matrix. The matrix can comprise one or several polymers including HPMC, PEO, PVA, etc. Blends of polymers of the same class having, e.g., different molecular weight or cross-linking, or viscosity, etc., can also be used. It also possible to include non-gelling polymers in a matrix blend, so long as the matrix provides some degree of gelation upon sustained exposure to water.

PEO is a preferred polymer matrix component in compositions of the present invention. While HPMC is believed to be a suitable matrix polymer, the use of PEO has been found to be especially suited for controlled release of lorazepam. PEO has the desired interaction with lorazepam such that the release can be slowed/delayed but also completed within the needed time span. This discovery is a separate aspect of the invention; namely that PEO is generally superior to HPMC and other matrix polymers in formulating controlled release of lorazepam, regardless of the release parameters. Accordingly, another embodiment of the invention is a controlled release tablet, which comprises 0.5 to 10 mg of lorazepam dispersed in a controlled release matrix, wherein the matrix comprises polyethylene oxide. The matrix may contain other polymers such as HPMC, Eudragit RSPO, etc., but the amount of PEO should be the greatest of any other gelling polymer, if present.

The PEO used in making controlled release lorazepam tablets typically has an average (approximate) molecular weight of at least 900,000 and usually not greater than 5,000,000, but is not necessarily limited thereto. Commercially available PEO, sold under the brand name POLYOX by The Dow Chemical Corp., have molecular weights up to 7,000,000. Higher molecular weights generally result in slower release rates. Higher concentrations of PEO also tend to decrease the rate of release. Thus, using more of a lower MW PEO may provide similar release as using less of a higher MW PEO. Typically the tablets are formed with PEO having an average MW between 900,000 and 4,000,000, more typically from 900,000 to about 2,000,000. If not commercially available, a desired MW can be achieved by blending commercially available PEO of different average MW. In one embodiment, the PEO has a MW of about 1,000,000 (e.g., +/−100,000).

The amount of polymer matrix in the tablet is typically from 20% to 70% of the tablet, more typically from 30% to 60% (all percentages for tablet ingredients refer to weight percent unless otherwise noted). As mentioned above, the polymer matrix is preferably composed entirely of PEO, but may be a combination of PEO and other matrix polymers so long as PEO is the single most prevalent type of matrix polymer. When the matrix polymer is primarily (or exclusively) PEO having a MW from 900,000 to 1,500,000, the amount of matrix polymer is often in the range of 35 to 55%, including 40% to 50%. Matrix polymer having higher MW PEO is generally used in slightly lower amounts such as 20% to 50%, including 25% to 45%.

In addition to the polymer matrix, a controlled release tablet usually contains other excipients including diluents and lubricants. Diluents provide bulk and can enhance tableting or tablet properties in comparison to the use of active and matrix polymer alone. Examples of diluents include sugars such as lactose or mannitol; microcrystalline cellulose; and calcium phosphates such as dibasic calcium phosphate dihydrate, dibasic calcium phosphate anhydrous, and tribasic calcium phosphate. Lubricants include magnesium stearate and sodium stearyl fumarate. Diluents often comprise from 30% to 70% of the tablet and can be a single diluent or a combination of diluents. Lubricants, when present, typically comprise 1% to 3% of the tablet weight. Because the dose of lorazepam is small (1-10 mg), the concentration of lorazepam tends to be low and typically is less than 10%, more typically less than 5%, and often in the 1-3% range. The combination of polymer matrix and diluent(s) is typically 85% to 99%, more typically 90% to 98%, and even 95% to 98%, the weight of the tablet.

In one embodiment, the tablet contains lactose and calcium phosphate as diluents. The lactose is typically lactose monohydrate and the calcium phosphate is typically dibasic calcium phosphate. The lactose is typically used in an amount from 10 to 50%, such as from 20 to 40%, but is not limited thereto. Likewise, the calcium phosphate is typically used in an amount from 10 to 50%, such as from 20 to 40%, but is not limited thereto. This dual diluent strategy is not limited to lactose and calcium phosphate as either one, or both, can be replaced with another diluent. But it is preferred that at least one of lactose and calcium phosphate, and more preferably both, are present in the tablet within the above ranges.

The tablets typically have a weight of 100 to 200 mg, not including the weight of any subsequent coatings. Generally any coating applied to the controlled release matrix tablet is for cosmetic or stability reasons and not for significant or meaningful release modification.

The tablets can be made by procedures known in the art. Wet granulation and direct compression are two common techniques for making tablets. Direct compression is usually preferred for economic reasons and is often more suited for gelling polymers such as PEO.

The controlled release tablets containing a PEO polymer matrix are preferably formulated (e.g., selection of PEO MW, amount of PEO, etc.) to achieve the above-described in vitro dissolution release parameters of substantially zero order release and 90% release within 7 to 12 hours, and/or the other release parameters as fully described above.

The pharmaceutical composition of the invention that achieves substantially zero order release and provides 90% release within 7 to 12 hours, preferably provides therapeutic effect for 24 hours under steady state conditions with daily dosing. Quantitatively, preferred embodiments of the invention will provide a blood plasma concentration of 10 ng/ml or more for at least 20 hours, often at least 22 hours, and sometimes for 24 hours under steady state conditions over a 24 hour period (e.g., from daily-dose to daily-dose). For clarity, the term “steady state” is used in its ordinary sense in the pharmaceutical arts. It does not mean constant, but rather the dynamic equilibrium that is obtained after consistent successive administrations of a drug, typically several days (e.g., 5 times the ½ life, or 3-5 days in the case of lorazepam). For example, a patient already taking lorazepam immediate release tablets on a regular schedule (two or three times per day) has lorazepam in his/her blood when the next dose is administered. After ingestion, the dose is released and the amount of lorazepam in the blood increases to a maximum blood plasma concentration or “Cmax.” The lorazepam is concurrently being metabolized and/or removed from the blood by biological actions of the body and so the blood plasma concentration falls. The decline in drug blood plasma concentration will continue until the next dose of lorazepam is taken. The drug blood plasma concentration will reach its lowest concentration level, the “Cmin,” just before the new dose of lorazepam is absorbed into the blood. The new dose causes a rise in blood plasma concentration and the cycle repeats, reaching the Cmax once again followed by a fall to the Cmin and a new administration of lorazepam, etc. In contrast to the steady state, the first dose of lorazepam produces different blood plasma values because no lorazepam is in the blood at the time of the dose. The Cmin for such a single dose experiment is zero at the outset. The Cmax is typically noticeably lower than the steady state Cmax. Because the present invention is applicable for chronic administration of lorazepam (one or more weeks and perhaps months or years), the steady state parameters can be more meaningful. Indeed, in some embodiments of the present invention, a single dose study (e.g., initial dose) will not provide a therapeutic concentration in the blood stream sooner than 1 hour, often not before 2 hours, and in some embodiments not before 3 hours. In some embodiments, a minimum therapeutic concentration can be taken as 10 ng/ml.

Preferred embodiments provide a steady state Cmax from daily dosing that is about equal to, or less than, the steady state Cmax obtained from dosing the same total daily amount of lorazepam via immediate release tablets through b.i.d or t.i.d. regimens. The term “about equal” means that the steady state Cmax of the controlled release composition is within +/−35%, preferably with +/−20% of the steady state Cmax for the corresponding immediate release tablets given b.i.d. or t.i.d (having the same total daily dose). In some embodiments, the steady state Cmax of the controlled release composition approximates, or is less than, the steady state Cmax for the corresponding immediate release tablets given b.i.d. The term “approximates” means +/−15%, preferably +/−10%. The steady state Cmin for the controlled release composition is preferably about equal to, or greater than, the steady state Cmin for the corresponding immediate release tablets given b.i.d. or t.i.d (having the same total daily dose). In some embodiments the steady state Cmin approximates or is greater than the Cmin for the corresponding immediate release tablets given b.i.d. The terms “about equal” and “approximates” have the same meaning as regards the Cmax.

Though the steady state Cmax and Cmin values of the inventive controlled release composition may be about equal to the Cmax and Cmin of the corresponding dose of immediate release tablets in b.i.d. (or t.i.d.), the controlled release composition is believed to lower the risk of adverse events. Beyond Cmax, the change in the blood plasma concentration is also thought to contribute to adverse events and/or the risk thereof. The controlled release composition of the present has less fluctuation in blood plasma concentration; i.e., one peak and trough per 24 hours, versus 2 or 3 peak-trough cycles for immediate release therapy. Moreover, the rate of increase of lorazepam blood concentration is believed to correlate with adverse event risk: a slower rise in lorazepam blood plasma concentration has less risk of adverse events. The composition of the present invention in a once-daily dose form provides a slower increase in lorazepam blood concentration than the immediate release tablets. This difference can be expressed by the “Tmax,” i.e., the time to regain steady state Cmax after a dose is administered. Preferred embodiments of the pharmaceutical composition of the invention typically provide a Tmax not sooner than 6 hours, more typically not sooner than 8 hours, and often not sooner than 10 hours. Conversely, the Tmax typically occurs not later than 14 hours, and usually not later than 13 hours. A Tmax of 12 hours+/−1 hour (or even +/−30 minutes) is generally preferred for once-daily dosing.

Specific embodiments of the present invention relate to a pharmaceutical composition that contains 2 mg of lorazepam and provides for once-daily dosing. Such a composition preferably provides a steady state Cmax of 26 ng/ml or less, usually 23 ng/ml or less when administered once daily. The Cmin, however, does not fall below therapeutic levels. Preferably the Cmin is at least 10 ng/ml, sometimes at least 11 ng/ml, and can be at least 12 ng/ml, when administered once daily. The Tmax is typically within the range of 10 to 14 hours after once-daily administration.

Typically the composition of the present invention exhibits dose proportionality within the range of 1-6 mg of lorazepam. The proportionality is typically with respect to the AUC (total exposure) but is also preferably found with the steady state Cmax. The following approximation can apply to preferred embodiments regarding the steady state Cmax. Each 1 mg of lorazepam provides a steady state Cmax of not greater than 10 ng/ml+20%. Thus under this embodiment, a 2 mg dose preferably provides a steady state Cmax within the range of 20 to 24 ng/ml or less; a 3 mg dose preferably provides a steady state Cmax within the range of 30 to 36 ng/ml or less, etc.

For clarity, all of the values for steady state Cmax, Cmin, and Tmax can be for a single subject but more commonly are an average of multiple subjects, e.g., multiple patients, multiple participants in a bioavailability study, etc. Also, the steady state values can be calculated from a single dose study by methods known in the art. Such calculated values are suitable for determining the steady state values for purposes of the present invention.

The pharmaceutical compositions of the invention can be used to treat any lorazepam-treatable condition. These conditions are most often related to the treatment or management of anxiety including anxiety related disorders. Examples include, but are not limited to: Generalized Anxiety Disorder and anxiety associated with major depression. But other uses for lorazepam can also apply to this invention; e.g., PTSD, insomnia and/or sleep disorders, bipolar disorder, obsessive-compulsive disorder (OCD), social anxiety disorder, convulsions, etc. The pharmaceutical compositions of the present invention are generally administered once per day. Though the dose is usually administered once daily, some clinicians may elect to divide the total daily dose amount for some patients into one or more administrations per day.

The invention will be further described with respect to the following non-limiting example.

### Example

Pharmaceutical tablets having a PEO matrix were made having the following nominal composition.

| | Reference | | Unit | |
|---|---|---|---|---|
| | to Quality | | Composition | |
| Component | Standards | Function | mg/Tablet | % w/w |
| Lorazepam | USP | Active | 2.0 | 1.35 |
| | | ingredient | | |
| Calcium hydrogen | USP | Filler/ | 38.5 | 25.65 |
| phosphate dihydrate | | Compression | | |
| (EMCOMPRESS ®) | | aid | | |
| Polyethylene | NF | Release | 67.5 | 45.00 |
| oxide (Polyox | | control | | |
| WSR N-12K) | | agent | | |
| Lactose | USP/NF | Filler | 40.5 | 27.00 |
| monohydrate | | | | |
| Magnesium | USP/NF | Lubricant | 1.5 | 1 |
| stearate | | | | |
| Total | | | 150.0 | 100.0 |

USP = United States Pharmacopeia.
NF = National Formulary.

The tablets are typically made by screening lorazepam (20 mesh sieve) and the excipients (30 mesh sieve), except magnesium stearate, and mixing in a V-blender for approximately 20 minutes in total at 25 RPM. To the powder mixture is added the magnesium stearate and mixed for approximately 2 minutes. The resulting blend is tableted using 8 mm round standard concave punches/dies to form a batch of 6500 lorazepam tablets.

The dissolution of the tablets is measured by subjecting a sample tablets to an in vitro dissolution test. The conditions and typical results are shown below.

| | | | |
|---|---|---|---|
| Apparatus | USP Apparatus 2 | Volume | 500 mL |
| Rotation Speed | 50 RPM | Temperature | 37° C. ± 0.5° C. |
| Medium | pH 6.8 Phosphate | Number of Units | 3 |
| | Buffer | | |

| | | Collection Time (h) | | | | | | |
|---|---|---|---|---|---|---|---|---|
| | | 1 | 2 | 4 | 6 | 8 | 10 | 12 |
| Percent | Mean | 12 | 27 | 52 | 73 | 90 | 94 | 97 |
| Dissolved | Min | 11 | 25 | 48 | 68 | 85 | 92 | 96 |
| | Max | 14 | 29 | 55 | 76 | 93 | 97 | 98 |
| | RSD | 12.4 | 7.4 | 6.9 | 6.0 | 4.5 | 2.7 | 1.0 |

RSD = Relative Standard Deviation.

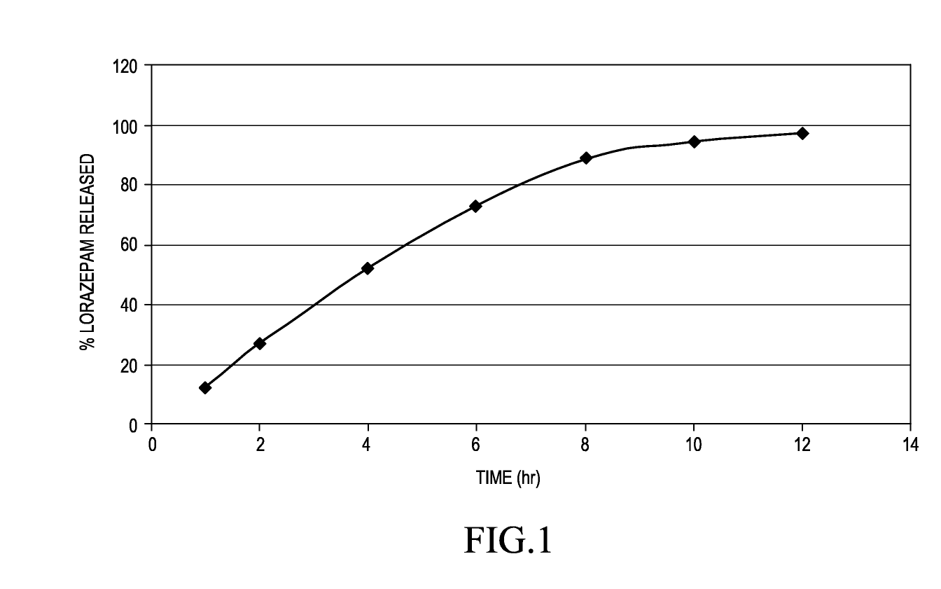

The above pharmaceutical dissolution test shows that the tablets have a substantially zero order release and reach 90% release at 8 hours. The mean dissolution values are plotted in FIG. 1.

Each of the patents and articles mentioned above are incorporated herein by reference. The invention having been described it will be obvious that the same may be varied in many ways and all such modifications are contemplated as being within the scope of the invention as defined by the following claims

1. A pharmaceutical composition, comprising 0.5 to 10 mg of lorazepam in combination with sufficient pharmaceutically acceptable excipients to provide a solid oral dosage form having controlled release of said lorazepam; wherein said controlled release of lorazepam is (1) substantially zero order release and (2) the release of lorazepam reaches 90% within the time range of 7 to 12 hours; and wherein said controlled release parameters are determined in a pharmaceutical dissolution test comprising a buffer of pH 6.8.

2. The pharmaceutical composition according to claim 1, wherein said 90% release of lorazepam occurs within 7.5 to 11 hours.

3. The pharmaceutical composition according to claim 1, wherein said controlled release of lorazepam releases not more than 40% of the lorazepam at two hours.

4. The pharmaceutical composition according to claim 1, wherein said composition, when administered once daily to a patient, maintains a therapeutic effect for at least 24 hours.

5. The pharmaceutical composition according to claim 1, wherein said composition contains 1 to 4 mg of lorazepam.

6. The pharmaceutical composition according to claim 1, wherein said composition provides a Tmax within the range of 6 to 14 hours when administered once daily.

7. The pharmaceutical composition according to claim 1, wherein said composition provides a steady state Cmax that is about equal to or less than the corresponding Cmax achieved by immediate release tablets given b.i.d. and having the same total daily dose.

8. The pharmaceutical composition according to claim 1, wherein said composition provides a steady state Cmin of at least 10 ng/ml when administered once daily.

9. The pharmaceutical composition according to claim 1, wherein said composition contains 2 mg of lorazepam, provides an average steady state Cmax of 26 ng/ml or less when administered once daily, provides an average steady state Cmin of at least 10 ng/ml when administered once daily, and provides an average steady state Tmax within the range of 10 to 14 hours after once daily administration.

10. The pharmaceutical composition according to claim 1, wherein said excipients include a gelling polymer and said lorazepam is dispersed in said gelling polymer matrix.

11. The pharmaceutical composition according to claim 10, wherein said gelling polymer is polyethylene oxide.

12. The pharmaceutical composition according to claim 11, wherein said polyethylene oxide has a molecular weight of at least about 900,000.

13. The pharmaceutical composition according to claim 12, wherein said polyethylene oxide comprises 20 to 70% by weight of said composition.

14. The pharmaceutical composition according to claim 11, wherein said lorazepam is contained in an amount of 1 to 3%.

15. The pharmaceutical composition according to claim 11, wherein said excipients further comprise 10 to 50% of a lactose.

16. The pharmaceutical composition according to claim 15, wherein said excipients further comprises 10 to 50% of a calcium phosphate.

17. (canceled)

18. The controlled release tablet according to claim 12, wherein said polyethylene oxide has an average molecular weight of 900,000 to 2,000,000.

19. A method of treating a lorazepam-treatable condition in a patient, which comprises administering once a day to a patient in need thereof a pharmaceutical composition according to claim 1 that provides 24 hour therapeutic effect during steady state conditions.

20. (canceled)
